(19) United States
(12) Patent Application Publication
(10) Pub. No.: US 2005/0143450 A1
(43) Pub. Date: Jun. 30, 2005

(54) Alkyl PCDF as a treatment for prostate cancer

(75) Inventor: Stephen H. Safe, College Station, TX (US)

Correspondence Address: FULBRIGHT & JAWORSKI, LLP, 1301 MCKINNEY, SUITE 5100, HOUSTON, TX, 77010-3095, US

(73) Assignee: Texas A&M University System, College Station, TX (US)

(21) Appl. No.: 10/994,795

(22) Filed: Nov. 22, 2004

Related U.S. Application Data
(63) Continuation of application No. PCT/US03/15764, filed on May 20, 2003.
(60) Provisional application No. 60/382,693, filed on May 22, 2002.

(51) Int. Cl. A61K 31/343

(52) U.S. Cl. 514/468

(57) ABSTRACT

The present invention is directed to a method of modulating a prostate neoplasm by administering an alkyl-substituted PCDF.

This application is a Continuation Application of International Application No. PCT/US03/15764 filed on May 20, 2003, which claims priority to U.S. Provisional Application No. 60/382,693 filed on May 22, 2002, each of which is incorporated herein by reference.

This invention was made with government support under NIH Grant No. ES09106 awarded by the National Institutes of Health and DAMD17-02-0147 awarded by the Department of Defense Army Medical Research and Material Command. The United States Government may have certain rights in the invention.

### FIELD OF INVENTION

This invention relates to tumor therapy and prevention. More particularly, it relates to methods for modulating a prostate neoplasm.

### BACKGROUND OF THE INVENTION

Prostate cancer is the most commonly diagnosed cancer in North American men and it is estimated that there are over 300,000 newly diagnosed cases each year (Landis, et al., 1998; Shibata, et al., 1998). The incidence and mortality rates from prostate cancer are increasing and this is due, in part, to an increasingly aging population and the higher incidence of this disease in older men (Gao et al., 1997; Chiarodo, 1991). Both benign prostatic hypertrophy (BPH) and prostate cancer are decreased or not detected in eunuchs and are linked not only to advancing age but the presence of testes and androgen function (Gao et al., 1997; Chiarodo, 1991; Sakti and Crawford, 1993).

Early prostate cancer tends to be androgen-dependent and requires expression of a functional androgen receptor (AR), whereas later stage tumors progress to androgen-independence which in some cases is correlated with loss of AR function (Cheng et al., 1993). Interestingly, the progression from early stage hormone-dependent to latter stage hormone-independence in prostate cancer in men is also observed for breast cancer in women where estrogen-responsiveness undergoes a similar pattern of change in women with early or late stage disease (Hopp and Fuqua, 1999; Fuqua et al., 1995).

Prostate cancer therapy is dependent on the stage of the tumor and AR expression. Early stage androgen-responsive prostate cancers can be treated by castration or with antiandrogens or drugs that block androgen-induced responses including steroidal antiandrogens (cyproterone), LHRH analogs, nonsteroidal antiandrogens (flutamide, nilutamide, bicalutamide), and the potent estrogenic drug diethylstilbestrol (reviewed in (Sadar et al., 1999; Klotz, 2000; Morris et al., 2000; Boccardo, 2000). In addition, there are several possible novel strategies for treatment of prostate cancer and other tumor-types and these include targeting of critical genes involved in tumor cell growth and metastasis (e.g., antiangiogenic drugs, antisense therapy) (Boasberg et al., 1997; Knox et al., 1998; 1998; Yamaoka et al., 1993; Folkman, 1995; Folkman, 1971). Ligands for nuclear receptors (NR) are also being developed for treatment of prostate cancer through inhibitory NR-AR crosstalk that involves various ligands or drugs that bind the retinoid acid/X-receptors (retinoids), vitamin D receptor (calcitrol), and peroxisome proliferator activate receptor γ (trogilatazone) (Dorai et al., 1997; Pienta et al., 1993; Pollard et al., 1991; Kelly et al., 1996; Miller et al., 1992; Miller et al., 1995; Peehl et al., 1994; Gross et al., 1998; Kubota et al., 1998; Tontonoz et al., 1997; Tontonoz et al., 1994; Smith et al., 1999).

The present invention is the first to utilize alkyl-substituted polychlorinated dibenzofurans (PCDFs) as new chemotherapy for the treatment of prostate cancer.

### BRIEF SUMMARY OF THE INVENTION

This invention relates to tumor therapy and prevention. More particularly, it relates to methods for modulating a prostate neoplasm. It is envisioned that the present invention can be used to treat a subject suffering from an androgen responsive neoplasm or an androgen nonresponsive neoplasm. Yet further, it is contemplated that the compounds used in the present invention are ligands for the aryl hydrocarbon receptor (AhR). It is envisioned that AhR agonists used in the present invention will undergo inhibitory AhR crosstalk with hormone receptors leading to modulation of a prostate neoplasm. Thus, the AhR agonsits are selective AhR modulators (SAhRMs).

The present invention provides a method of modulating a prostate neoplasm comprising administering to a subject an effective amount of a compound of the formula: 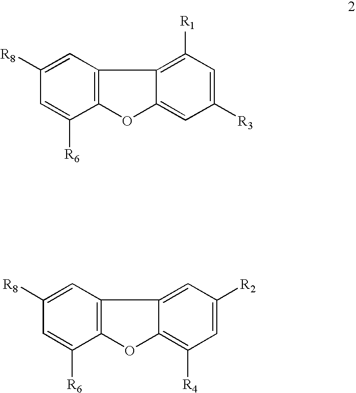
or

Given the above formula, R₁, R₃, R₆ and R₈ or R₂, R₄, R₆ and R₈ are individually and independently a hydrogen or a substituent selected from the group consisting of chlorine, fluorine, and bromine, and a linear or branched alkyl group of one to four carbons. Preferably, the compound contains at least one alkyl substituent and at least two halogen substituents. More preferably, the halogen substituents are selected from the group consisting of chlorine, bromine, and fluorine and the alkyl substituents are selected from the group consisting of methyl, ethyl and propyl.

In specific embodiments, the compound can be selected from the following 6-methyl-1,3,8-trichlorodibenzofuran, 8-methyl-1,3,6-trichlorodibenzofuran, 6-ethyl-1,3,8-trichlorodibenzofuran, 6-propyl-1,3-8-trichlorodibenzofuran, 6-methyl-2,3,8-trichlorodibenzofuran, 6-methyl-2,3,4,8-tetrachlorodibenzofuran, 8-methyl-1,3,7-trichlorodibenzofuran, 8-methyl-1,2,4,7-tetrachlorodibenzofuran, 8-methyl-2,3,7-trichlorodibenzofuran, or 8-methyl-2,3,4,7-tetrachlorodibenzofuran.

Another embodiment is a method of treating androgen-dependent or androgen-independent tumors comprising administering to a subject in need of such treatment an effective amount 6-methyl-1,3,8-trichlorodibenzofuran.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1A and FIG. 1B show induction of EROD activity in prostate cancer cell lines. FIG. 1A show PC3 cells treated with 2,3,7,8-tetrachloro-p-dioxin (TCDD) for 24, 48, 72 and 96 hr. FIG. 1B show 22Rv1 cells treated with TCDD for 24 hr.

FIG. 2A and FIG. 2B show the growth inhibitory effects of TCDD and 6-methyl-1,3,8-trichlorodibenzofuran (6-MCDF) on prostate cancer cells. FIG. 2A shows the inhibition of growth of 22Rv1 cells by TCDD. FIG. 2B shows the inhibition of growth of 22Rv1 cells by 6-MCDF.

FIG. 3A, FIG. 3B, and FIG. 3C show growth inhibition in LNCaP cells. FIG. 3A ligand-dependent AhR activation and growth inhibition in LNCaP cells. FIG. 3B shows growth of LNCaP cells in absence of 10 nM DHT and FIG. 3C shows growth of LNCaP cells of 10 nM DHT.

FIG. 4A and FIG. 4B show inhibition of AR-dependent transactivation by TCDD and 6-MCDF. LNCAP cells were transfected with pPB (FIG. 4A) or pARR3 (FIG. 4B), treated with hormone or AhR agonist alone or in combination.

FIG. 5A, FIG. 5B, and FIG. 5C show inhibition of AR-dependent transactivation by antiandrogens and antiestrogens in LNCaP cells. Cells were transfected with pPB (FIG. 5A), pARR3 (FIG. 5B) or pPB (FIG. 5C), treated with various compounds.

FIG. 6A and FIG. 6B show inhibition of hormone-induced transactivation in ZR-75 breast cancer cells transfected with pPB. FIG. 6A shows transfection with pPB alone and FIG. 6B shows transfection with pPB and hAR.

FIG. 7A and FIG. 7B show AR protein expression in LNCaP cells treated with hormones, AhR agonists, antiandrogens and antiestrogens. In FIG. 7A, LNCaP cells were treated with DHT, E2, TCDD, 6-MCDF and their combinations for 6 h, and AR protein levels in whole cell lysates were determined by Western blot analysis. p27 protein was determined. In FIG. 7B, AR protein levels were determined as in FIG. 7A and blots were stripped and reprobed with cyclin D1 antibodies.

### DETAILED DESCRIPTION OF THE INVENTION

It is readily apparent to one skilled in the art that various embodiments and modifications can be made to the invention disclosed in this Application without departing from the scope and spirit of the invention.

A. Definitions

As used herein, the use of the word “a” or “an” when used in conjunction with the term “comprising” in the claims and/or the specification may mean “one,” but it is also consistent with the meaning of “one or more,” “at least one,” and “one or more than one.”

The term “androgen” as used herein refers to an agent that is typically a hormone (e.g., androsterone, testosterone) that stimulates activity of the male sex organs, encourages development of the male sex characteristics, etc. Androgens that are used in the present invention can be natural or native androgens, synthetic androgens or derivatives of androgens.

The term “androgen responsive” refers to a neoplasm that utilizes an androgen or a derivative thereof to develop, proliferative and/or metastasize. Yet further, as used herein, the terms “androgen responsive” and “androgen-dependent” are interchangeable.

The term “effective amount” as used herein is defined as an amount of the agent that will decrease, reduce, inhibit or otherwise abrogate the growth of a neoplasm, induce apoptosis, inhibit angiogenesis of a neoplasm, inhibit metastasis, or induce cytotoxicity in a neoplasm. Thus, an effective amount is an amount sufficient to detectably and repeatedly ameliorate, reduce, minimize or limit the extent of the disease or its symptoms.

The term “modulate” as used herein refers to the suppression, enhancement, or induction of a function. More specifically, “modulate” or “regulate” also refers to methods, conditions, or agents which increase or decrease the biological activity of a protein, enzyme, inhibitor, signal transducer, receptor, transcription activator, co-factor, and the like. Such enhancement or inhibition may be contingent upon occurrence of a specific event, such as activation of a signal transduction pathway and/or may be manifest only in particular cell types. In specific embodiments, modulate refers to increasing or decreasing the ability of a cell to proliferate, for example a hyperproliferative cell or a neoplasm cell, etc.

The term “prostate” as used herein refers to the structure that surrounds the upper part of the urethra in a male or female.

The term “neoplasm” as used herein refers to an abnormal formation of tissue, for example, a tumor. One of skill in the art realizes that a neoplasm encompasses benign tumors and/or malignant tumors. Yet further, as used herein the terms “neoplasm” and “tumor” are interchangeable.

The term “non-androgen responsive” refers to a neoplasm that does not utilize an androgen or a derivative thereof to develop, proliferative and/or metastasize. Yet further, as used herein, the terms “non-androgen responsive” and “androgen-independent” are interchangeable.

The term “subject” as used herein, is taken to mean any mammalian subject to which a composition of the present invention is administered according to the methods described herein. In a specific embodiment, the methods of the present invention are employed to treat a human subject. Another embodiment includes treating a human subject suffering from a prostate neoplasm.

The term “therapeutically effective amount” as used herein refers to an amount that results in an improvement or remediation of the symptoms of the disease or condition.

The term “treating” and “treatment” as used herein refers to administering to a subject a therapeutically effective amount of an alkyl substituted PCDF so that the subject has an improvement in the disease. The improvement is any improvement or remediation of the symptoms. The improvement is an observable or measurable improvement. Thus, one of skill in the art realizes that a treatment may improve the disease condition, but may not be a complete cure for the disease.

B. Treatment of Prostate Neoplasms

In certain embodiments, a subject suffering from a prostate neoplasm may be treated by administering to the subject an effective amount of an alkyl-substituted polychlorinated dibenzofurans (PCDFs). The subject is preferably a mammal and more preferably a human.

The alkyl-substituted PCDFs that are used in the present invention are ligands for the aryl hydrocarbon receptor (AhR). It is envisioned that these alkyl-substituted PCDFs used in the present invention undergo inhibitory AhR crosstalk with hormone receptors leading to modulation of a prostate neoplasm. Thus, the alkyl-substitutued PCDFs of the present invention are selective AhR modulators (SAhRMs).

Yet further, it is contemplated that neoplasms that metastasize may be treated in the present invention. It is understood by those in the art that metastasis is the spread of cells from a primary tumor to a noncontiguous site, usually via the bloodstream or lymphatics, which results in the establishment of a secondary tumor growth.

1. Compounds Used for Treatment

The compounds utilized in present invention have are 1,3,6,8-substituted or 2,4,6,8-substituted alkyl PCDF. The possible substituents include halogens such as bromine, chlorine, fluorine and/or linear or branched substituents such as alkyl groups of about one to about five carbons. The 2, 4, 6 or 8 and 1, 3, 6 or 8 positions may also be individually and independently occupied by a hydrogen instead of a substituent. Suitable alkyl substituents include, but are not limited to, methyl, ethyl, propyl, isopropyl (i-propyl), n-butyl, sec-butyl, or tert-butyl groups. It is envisioned that the PCDF in the present invention contain at least one alkyl substituent, however, it is well within the scope of the present invention that the PCDF may contain two or more alkyl substitutents.

The PCDFs used in the present invention are described, for example, in U.S. Pat. No. 5,516,790, issued to Stephen Safe on May 14, 1996, which is hereby incorporated by reference herein in its entirety. The PCDFs may include, but are not limited to, those having the formula:

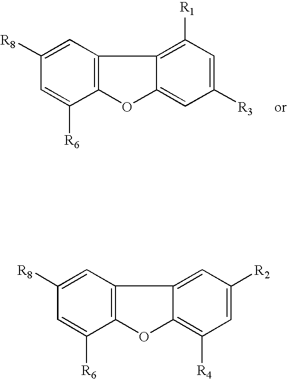

R₁, R₃, R₆ and R₈ or R₂, R₄, R₆ and R₈ are individually and independently a hydrogen or a substituent selected from the group consisting of chlorine, fluorine and bromine, and a linear or branched alkyl group of one to four carbons, and wherein the compound has at least one alkyl substituent and at least two halogen substituents; furthermore, the halogen may be chlorine, the alkyl substituent may be selected from the group consisting of methyl, ethyl and propyl; R₆ may be an alkyl substituent and R₁, R₃, and R₈ may be selected from the group consisting of chlorine, fluorine and bromine; further still R₈ may be an alkyl substituent and R1, R₃, and R₆ may be selected from the group consisting of chlorine, fluorine and bromine, the alkly substituent may be methyl; still further R₆ may be an alkyl and R₂, R₄, and R₈ may be selected from the group consisting of chlorine, fluorine and bromine; and, for example, R₈ may be an alkyl substituent and R₂, R₄, and R₆ may be selected from the group consisting of chlorine, fluorine and bromine.

Exemplary compounds included, but are not limited to 6-methyl-1,3,8-trichlorodibenzofuran (6-MCDF); 8-methyl-1,3,6-trichlorodibenzofuran (8-MCDF); 6-ethyl-1,3,8-trichlorodibenzofuran (6-ethyl-1,3,8-triCDF); -6-n-propyl-1,3-8-trichlorodibenzofuran (6-n-propyl-1,3-8-triCDF); 6-1-propyl-1,3-8-trichlorodibenzofuran (6-1-propyl-1,3-8-triCDF); 6-methyl-2,3,8-trichlorodibenzofuran (6-methyl-2,3,8-triCDF); 6-methyl-2,3,4,8-tetrachlorodibenzofuran (6-methyl-2,3,4,8-tetraCDF); 8-methyl-1,3,7-trichlorodibenzofuran (8-methyl-1,3,7-triCDF); 8-methyl-1,2,4,7-tetrachlorodibenzofuran (8-methyl-1,2,4,7-tetraCDF); 8-methyl-2,3,7-trichlorodibenzofuran (8-methyl-2,3,7-triCDF); 8-methyl-2,3,4,7-tetrachlorodibenzofuran (8-methyl-2,3,4,7-tetraCDF); and 8-methyl-2,3,7-trichlorodibenzo-p-dioxin and 8-methyl-2,3,7-tribromodibenzo-p-dioxin.

2. Treatment Regimens

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

Preferably, patients to be treated have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

To kill neoplasm cells, inhibit neoplasm cell growth, inhibit metastasis, decrease neoplasm or tissue size and otherwise reverse or reduce the malignant phenotype of neoplasm cells, using the methods and compositions of the present invention, one would generally contact a neoplasm with an effective amount of the alkyl-substituted PCDF of the present invention. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

The effective amount of alkyl-substituted PCDF that is used in the present invention is the amount that will decrease, reduce, inhibit or otherwise abrogate the growth of a neoplasm, induce apoptosis, inhibit angiogenesis of a neoplasm, inhibit metastasis, or induce cytotoxicity in a neoplasm. Thus, an effective amount is an amount sufficient to detectably and repeatedly ameliorate, reduce, minimize or limit the extent of the disease or its symptoms.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising the alkyl-substituted PCDF. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the alkyl-substituted PCDF composition via continuous perfusion will be equivalent to that given by a single or multiple injections, adjusted over a period of time during which the perfusion occurs.

In certain embodiments, the tumor being treated may not, at least initially, be resectable. Treatments with alkyl-substituted PCDF may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of an effective amount of the alkyl-substituted PCDF. The quantity to be administered, and the particular route and formulation, are within the skill of those in the clinical arts. A unit dose need not be administered as a single injection but may comprise continuous infusion over a set period of time.

C. Combination Treatments

In order to increase the effectiveness of the alkyl-substituted PCDF, it may be desirable to combine the alkyl-substituted PCDF with other agents effective in the treatment of prostate tumors, such as anti-cancer agents, or with surgery. It is also contemplated the alkyl-substituted PCDF may be administered in combination with an additional anti-cancer agent. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the tumor cell or growth of the tumor. This process may involve contacting the cells with alkyl-substituted PCDF and the anti-cancer agent(s) or multiple factor(s) at the same time. This may be achieved by contacting the cell with a single composition or pharmacological formulation that includes both the alkyl-substituted PCDF and the anti-cancer agent, or by contacting the cell with two distinct compositions or formulations, at the same time, wherein one composition includes the alkyl-substituted PCDF and the other includes the second agent(s) or anti-cancer agent.

Alternatively, the alkyl-substituted PCDF of the present invention may precede or follow the other anti-cancer agent treatment by intervals ranging from minutes to weeks. In embodiments where the other anti-cancer agent and alkyl-substituted PCDF are applied separately to the cell, one would generally ensure that a significant period of time did not expire between the time of each delivery, such that the agent and alkyl-substituted PCDF would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, alkyl-substituted PCDF is “A” and the secondary agent, such as an anti-cancer agent or anti-cancer therapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

1. Chemotherapy

Cancer therapies also include a variety of chemical based treatments. Some examples of chemotherapeutic agents include antibiotic chemotherapeutics such as Doxorubicin, Daunorubicin, Adriamycin, Mitomycin (also known as mutamycin and/or mitomycin-C), Actinomycin D (Dactinomycin), Bleomycin, Plicomycin, plant alkaloids such as Taxol, Vincristine, Vinblastine, miscellaneous agents such as Cisplatin (CDDP), etoposide (VP16), Tumor Necrosis Factor, and alkylating agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine.

Some examples of other agents include, but are not limited to, Estramustine, Taxol, Paclitacel, Docetaxel, Gemcitabien, Navelbine, Farnesyl-protein transferase inhibitors, Transplatinum, 5-Fluorouracil, hydrogen peroxide, and Methotrexate, Temazolomide (an aqueous form of DTIC), Dolastatin-10, Bryostatin, or any analog or derivative variant of the foregoing.

2. Radiotherapeutic Agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

4. Gene Therapy

In yet another embodiment, gene therapy in conjunction with the combination therapy using the alkyl-substituted PCDF described in the invention are contemplated. Various genes that may be targeted for gene therapy of some form in combination with the present invention included but are not limited to cell cycle genes, i.e., cyclin E, cyclin A, cdk4, cdk2, ckd6, cdk7, cyclin H, cdc25A, p21, p27, and retinoblastoma (Rb).

5. Biological Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Adjuvant therapy may also be used in conjunction with the present invention. The use of adjuvants or immunomodulatory agents include, but are not limited to tumor necrosis factor; interferon alpha, beta, and gamma; IL-2 and other cytokines; F42K and other cytokine analogs; or MIP-1, MIP-1beta, MCP-1, RANTES, and other chemokines.

6. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

It is contemplated that vaccines that are used to treat cancer may be used in combination with the present invention to improve the therapeutic efficacy of the treatment. Such vaccines include peptide vaccines or dendritic cell vaccines. Peptide vaccines may include any tumor-specific antigen that is recognized by cytolytic T lymphocytes. Yet further, one skilled in the art realizes that dendritic cell vaccination comprises dendritic cells that are pulsed with a peptide or antigen and the pulsed dendritic cells are administered to the patient.

7. Hormonal Therapy

Hormonal therapy may also be used in conjunction with the present invention. Compounds that are known to be antiandrogens or block androgen-induced responses may be used in combination with the alkyl-substituted PCDFs of the present invention. For example, antiandrogens include steroidal antiandrogens (i.e., cyproterone, luteinizing hormone-releasing hormone analogs) and nonsteroidal antiandrogens (i.e., flutamide, nilutamide, bicalutamide). Other hormonal treatments may also include estrogenic drugs, for example, diethylstilbestrol.

D. Pharmaceutical Formulations and Delivery

The alkyl-substituted PCDFs disclosed herein may be administered parenterally, intravenously, intradermally, intramuscularly, transdermally intratumorally or even intraperitoneally as described in U.S. Pat. No. 5,543,158; U.S. Pat. No. 5,641,515 and U.S. Pat. No. 5,399,363 (each specifically incorporated herein by reference in its entirety).

Solutions of the alkyl-substituted PCDFs as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifingal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating alkyl-substituted PCDFs in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Transient Transfection Assays

ZR-75 human breast cancer and LNCaP human prostate cancer cells were obtained from American Type Culture Collection (Manassas, Va.) and were maintained in RPMI 1640 medium supplemented with 10% FBS, 1% antibiotic/antimycotic solution, 1.5 g/L sodium bicarbonate, and 10 mM HEPES, final pH of 7.4. Cells were seeded at 2.75×10⁵ per 22-mm well in DME-F12 without phenol red, supplemented with 2.5% charcoal-stripped fetal bovine serum (FBS). After 24 h, cells were transfected using Lipofectamine and Plus reagents (Invitrogen) according to manufacturer's instructions. LNCaP and ZR-75 cells were transfected with 500 ng/well of either reporter plasmid, and 250 ng/well of pcDNA3.1-β-gal (Invitrogen) as the internal control. In addition, ZR-75 cells were transfected with 500 ng hAR. Twenty-four hours after treatment, cells were harvested by scraping with 200 μL/well of reporter lysis buffer. Lysates were centrifuged at 40,000 g and luciferase and β-galactosidase activity was assayed with 30 μL of the supernatant extract per sample using a Lumicount luminometer (PerkinElmer, Boston, Mass.). Luciferase activity was normalized to β-galactosidase activity for each transfection well.

### Example 2

### Cell Proliferation Assay

After trypsinization and low-speed centrifugation, LNCaP cells were resuspended and counted using a Coulter cell counter (Beckman Coulter, Fullerton, Calif.). Cells were seeded at a density of 5×10⁴/35-mm well using DME-F12 without phenol red, supplemented with 2.5% charcoal-stripped fetal bovine serum (FBS). Twenty-four hours after seeding, initial treatment was applied and then subsequently reapplied with fresh medium every two days until harvesting by trypsinization. Cells were counted after harvesting using a Coulter counter.

### Example 3

### Fluorescence Activated Cell Sorting Analysis (FACS)

Cells were analyzed on a FACS Calibur (Becton Dickinson, San Jose, Calif.) flow cytometer, equipped with a 15 mW air-cooled argon laser, using CellQuest (Becton Dickinson) acquisition software. Propidium iodide (PI) fluorescence was collected through a 585/42-nm bandpass filter, and list mode data were acquired on a minimum of 12,000 single cells defined by a dot plot of PI-width versus PI-area. Data analysis was performed in ModFit LT (Verity Software House, Topsham, Me.) using PI-width versus PI-area to exclude cell aggregates.

### Example 4

### Western Immunoblot Analysis

Cells were harvested 6 h after treatment using 200 μL/22-mm well of ice cold lysis buffer (50 mM HEPES, pH 7.5, 500 mM NaCl, 10% (v/v) glycerol, 1% (v/v) Triton-X 100, 1.5 mM MgCl₂, 1 mM EGTA) (McDougal et al., 2001). Lysates were centrifuged at 40,000 g, and supernatant was collected as extract. Whole cell extracts (50 μg/sample) were separated by electrophoresis on a tiered 7.5% (top)/12.5% (bottom) SDS-polyacrylamide gel and transferred to PVDF membrane (Bio-Rad, Richmond, Calif.). The membrane was blocked with 5% milk (m/v) in tris-buffered saline 0.05% Tween (TBST). Membranes were incubated with primary antibodies for AR (sc-7305), cyclin D1 (sc-718), or p27 (sc-528) (each from Santa Cruz Biotechnology, Santa Cruz, Calif.) at 1:1000 in 5% milk/TBST for 3 h. Membranes were washed twice in TBST. Horseradish peroxidase (HRP)-conjugated secondary antibodies were applied at 1:5000 in 5% milk/TBST for 1 h. After two TBST washes, PVDF-bounded antibodies were detected using a chemiluminescence kit (Western Lightning, PerkinElmer), ImageTek-H film (American X-Ray and Medical Supply, Rancho Cordova, Calif.) and an autoprocessor (Hope Macro-Med, Warminster, Pa.). Quantitation of the Western blot was performed using a Sharp JX-330 scanner (Sharp, Mahwah, N.J.) and Zero-D software (Scanalytics, Billerica, Mass.).

### Example 5

### Characterization of AhR Expression and Function

An androgen responsive prostate cancer cell line 22Rv1 and an androgen nonresponsive prostate cancer cell line PC3 were treated with TCDD and ethoxyresorufin O-deethylase (EROD) activity was measured.

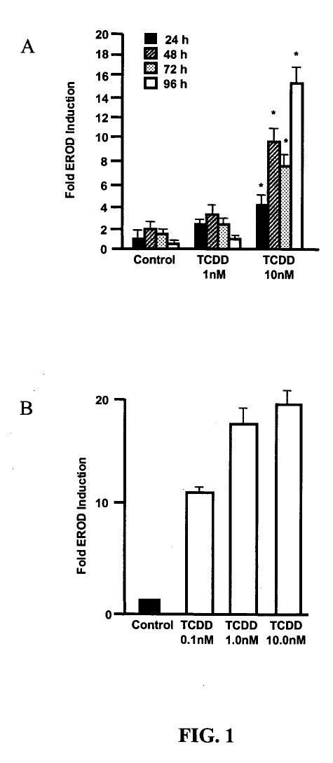

FIG. 1 shows that TCDD induced EROD activity in 22Rv1 cells over a 24-hr treatment period (FIG. 1A). Treatment of PC3 cells with 1 nM TCDD for 24-96 hr did not induce activity, whereas 10 nM TCDD induced a response which appeared to increase over time and was maximal after 96 hr (FIG. 1B). Thus, both 22Rv1 and PC3 cells were Ah-responsive.

### Example 6

### Growth-Inhibitory Effects of TCDD and 6-MCDF

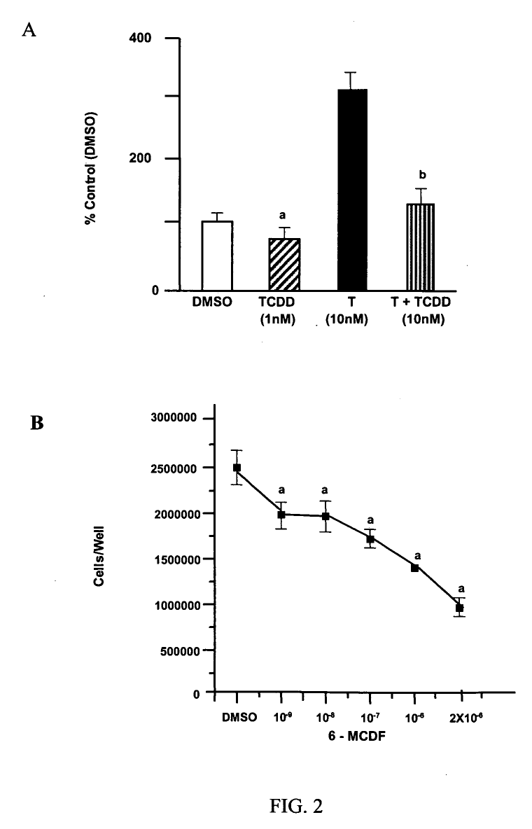

22Rv1 cells were treated with various concentrations of TCDD. FIG. 2A shows that 1 nM TCDD alone inhibited constitutive and testosterone-induced growth of these cells.

In a second study, 22Rv1 cells were maintained in 10% serum, and various concentrations of 6-MCDF were added to the cells. FIG. 2B shows that 6-MCDF induced a concentration-dependent decrease in cell proliferation.

Thus, these data show that selective AhR modulators (SAhRMs) were effective inhibitors of prostate cancer cell growth.

### Example 7

### Effects of TCDD and 6-MCDF on AhR Activation and Growth of LNCaP Cells

LNCaP cells were transfected with pDRE3, treated with various compounds and luciferase activity was determined as described in the above Examples.

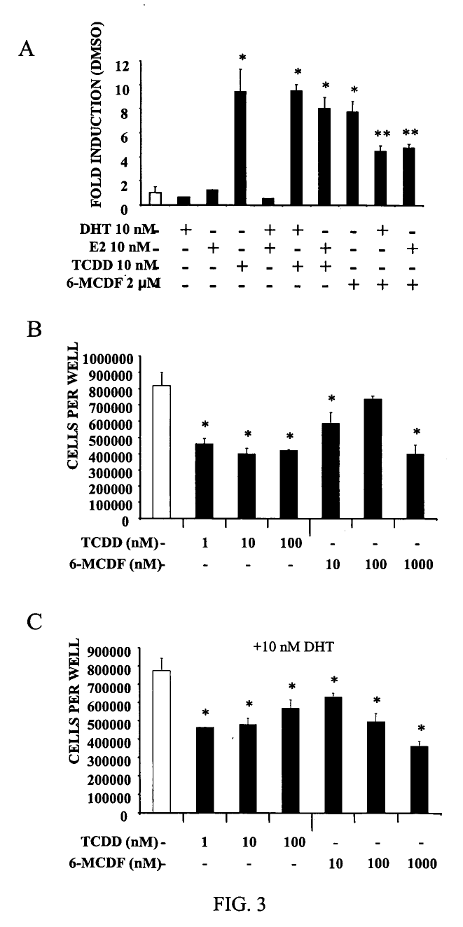

Results illustrated in FIG. 3A show that treatment of LNCaP cells with 10 nM TCDD induced luciferase activity >9-fold compared to solvent control (DMSO) in cells transfected with pDRE3. In contrast, 10 nM DHT (dihydrotestosterone), 10 nM E2 (17β-estradiol) and E2 plus DHT did not significantly induce activity, and neither DHT nor E2 in combination with TCDD affected induced activity. 6-MCDF (2 μM), a prototypical SAHRM, also induced luciferase activity (>7-fold). Both E2 and DHT in combination with 6-MCDF significantly inhibited 6-MCDF-induced activity, whereas in cells treated with TCDD in combination with E2 or DHT, inhibitory interactions were not observed.

Next, LNCaP cells were grown in the absence or presence of 10 nM DHT. Cells were cultured for six days, treated with different concentrations of TCDD or 6-MCDF in the absence or presence of 10 nM DHT and cell numbers were determined as described in the above Examples. The results showed that TCDD (1-100 nM) significantly inhibited proliferation of LNCaP cells, and growth inhibition was also observed for 6-MCDF (FIG. 3B). Both compounds inhibited >50% cell growth at one or more concentrations. Hormone-induced cell growth was not observed; however, both 6-MCDF and TCDD inhibited growth of LNCaP cells in the presence of DHT (FIG. 3C). These results confirmed that LNCaP cells were Ah-responsive and both TCDD and 6-MCDF inhibited LNCaP cell proliferation.

The effects of TCDD on cell cycle progression was also determined in LNCaP cells treated with 1.0, 10 and 100 nM TCDD for 48 h followed by FACS analysis (Table 1). The results showed that TCDD induced a small but significant increase in the percentage of cells in G₀/G₁ and a decrease of cells in S phase, whereas solvent (DMSO) and DHT (10 nM) exhibited minimal differences.

TABLE 1
Effects of TCDD on cell cycle progression
in LNCaP prostate cancer cells.^{a}

| | Percent Distribution | | |
|---|---|---|---|
| Treatment | G₀/G₁ | G₂/M | S |
| DMSO | 70.300 ± 1.779 | 10.973 ± 0.544 | 18.7 ± 1.258 |
| TCDD (10⁻⁹ M) | 74.300 ± 0.751* | 10.633 ± 0.376 | 14.7 ± 0.520* |
| TCDD (10⁻⁸ M) | 75.367 ± 0.636* | 10.300 ± 0.153 | 14.3 ± 0.666* |
| TCDD (10⁻⁷ M) | 77.500 ± 0.451* | 8.943 ± 0.471 | 13.567 ± 0.176* |
| DHT (10⁻⁸ M) | 73.400 ± 1.179 | 9.433 ± 1.011 | 17.167 ± 0.296 |

^{a}LNCaP cells were treated as indicated for 48 h and the percentage distribution of cells in G₀/G₁, G₂/M, and S phases were determined by FACS analysis.

### Example 8

### Inhibitory AhR-AR Crosstalk in LNCaP Cells Transfected with Androgen-Responsive Constructs

LNCaP cells were transfected with pPB that contained the −286 to +28 region of the androgen-responsive probasin gene promote. The transfected cells were then treated with hormone or AhR agonist alone or in combination and luciferase activity was determined as described in the above examples.

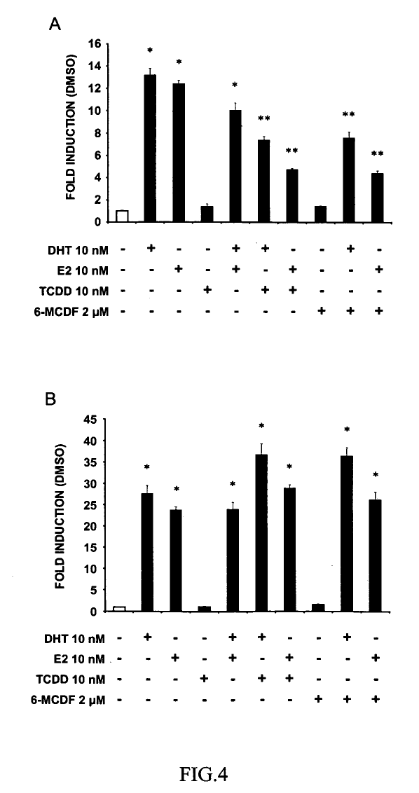

FIG. 4A shows that there was a >13-fold increase in luciferase activity in LNCaP cells treated with 10 nM DHT and transfected with pPB and the induced response was significantly inhibited after cotreatment with DHT plus TCDD. Similar inhibitory responses were also observed using 2 μM MCDF (FIG. 4A), whereas TCDD and MCDF alone did not significantly induce activity. Surprisingly, 10 nM E2 alone induced luciferase activity in LNCaP cells transfected with pPB, and the hormone-induced response was significantly decreased in cells cotreated with E2 plus TCDD or E2 plus 6-MCDF (FIG. 4A).

Next, LNCaP cells were transfected with pARR3 construct that contained three tandem (3) copies of the probasin androgen response element. The transfected cells were treated with hormone or AhR agonist alone or in combination and luciferase activity was determined as described in the above examples.

FIG. 4B shows that 10 nM DHT induced a >27-fold increase in luciferase in LNCaP cells transfected with pARR3; however, for this construct, cotreatment with DHT plus MCDF or TCDD did not decrease DHT-induced activity. E2 (10 nM) also induced luciferase activity (>24-fold) in cells transfected with pARR3 and in cells cotreated with E2 plus TCDD or MCDF, there was not a significant decrease in activity compared to that observed for E2 alone. These results confirmed that both DHT and E2 activated gene expression in cells transfected pPB or pARR3; however, inhibitory AhR-AR crosstalk was observed only for the pPB construct.

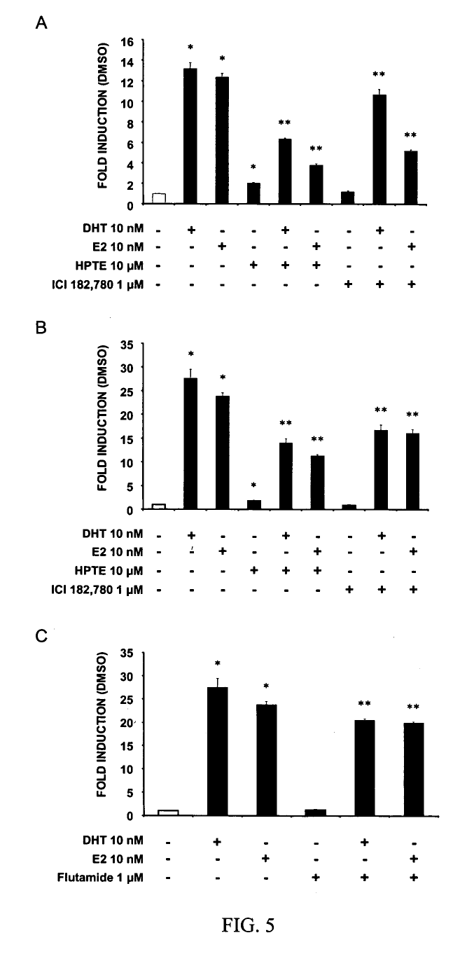

The comparative AR agonist activities of DHT and E2 were further investigated in LNCaP cells transfected with pPB and treated with hormones and antiandrogens or antiestrogens. Induction of luciferase activity by 10 nM DHT and E2 in LNCaP cells transfected with pPB was inhibited in cells cotreated with the hormone plus 10 μM HPTE, an AR antagonist (FIG. 5A). However, in parallel studies, the “pure” antiestrogen ICI 182780 also significantly inhibited E2-induced activity, whereas only minimal inhibition was observed in LNCaP cells treated with DHT plus ICI 182780. In a parallel experiment in LNCaP cells transfected with pARR3, both HPTE and ICI 182780 inhibited DHT and E2-induced luciferase activity (FIG. 5B), whereas 1 μM flutamide, an AR antagonist, caused only minimal decreases in hormone-induced activity (FIG. 5C).

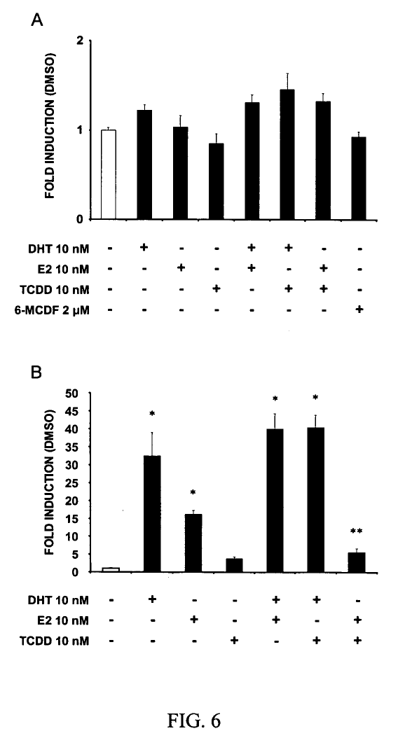

HPTE is also an ERα agonist and ERβ antagonist (Gaido et al., 2000) and the results obtained for both HPTE and ICI 182780 suggested a possible role for ERβ in mediating activation of pPB and pARR3. Results in FIG. 6A and FIG. 6B show that DHT, E2, TCDD and MCDF did not activate reporter gene activity in ZR-75 cells transfected with pPB alone; however, both DHT and E2 induced luciferase activity in cells cotransfected with pPB and hAR expression plasmid. Induction by E2 was significant but lower than observed for DHT in ZR-75 cells, and TCDD inhibited E2 but not DHT-induced activity in cells cotreated with hormone plus TCDD. Thus, the data confirmed that E2-dependent transactivation of pPB was androgen responsive.

### Example 9

### Effects of Various Treatments on AR, Cyclin D1 and p27 Protein Levels in LNCaP Cells

Levels of AR protein expression may influence androgen-responsiveness and inhibitory AhR-AR crosstalk.

LNCaP cells were treated with DHT, E2, TCDD, 6-MCDF and their combinations for 6 h, and AR protein levels in whole cell lysates were determined by Western blot analysis. p27 was essentially unchanged in all of the treatment groups and served as a loading control for this experiment.

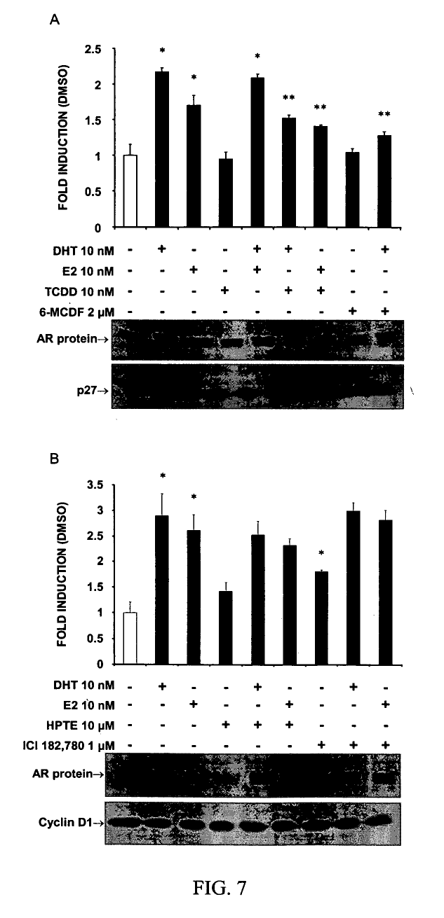

FIG. 7A shows that treatment with 10 nM DHT, 10 nM E2 or DHT plus E2 resulted in a significant increase in AR levels. In contrast, 10 nM TCDD and 2 μM 6-MCDF alone did not significantly affect levels of AR protein; however, in combination with DHT, there was a significant decrease in AR levels compared to cells treated with DHT alone. TCDD in combination with E2 also decreased AR levels compared to those observed in cells treated with E2 alone. In contrast, levels of immunoreactive p27 protein were not significantly changed by any of the treatments, and served as a loading control.

In a separate study, the effects of the antiandrogen HPTE and the antiestrogen ICI 182780 alone and in combination with E2 or DHT on AR levels were also determined (FIG. 7B). Ten μM HPTE alone did not affect AR levels in LNCAP cells, whereas ICI 182780 treatment increased AR levels compared to DMSO (solvent) treatment. Hormone (E2 or DHT)-induced upregulation of AR protein was not decreased cotreatment with HPTE or ICI 182780. Cyclin D1 protein was not significantly changed in this study and served as a loading control. These data demonstrated that various treatments differentially modulated AR protein levels in LNCaP cells.

### Example 10

### In Vivo Models for Inhibitory AhR-AR Crosstalk-Athymic Nude Mice

Athymic nude male BALB/c mice bearing xenografts of prostate cancer cell lines are used. The cells (LNCaP, PC-3, 22Rv1 or PCa2b) for the xenographs are obtained from the American Type Culture Collection (ATCC; Manassas, Va.) and are grown according to suggested protocols provided by ATCC. After the cells are grown, the cells are trypsinized, pelleted, and mixed 1:1 with Matrigel basement membrane. Each mouse receives two subcutaneous injection of 1.0×10⁶ cells, implanted dorsally in the flank region.

After allowing 14 days for tumor formation, animals bearing palpable tumors (200 mm³) are randomized into treatment groups and receive daily doses by gavage for 7 to 10 weeks and tumor size is assessed (2× per week) by measurement with calipers using the formula of length/2× width/2× height/2×π. The doses are administered via oral gavage. A range of SAhRMs (6-MCDF) in a concentration of 1 to 100 mg/kg/day are used to determine relative SAhRM potencies. The antiandrogen finasteride serves as a positive control inhibitor of tumor growth in animals bearing androgen-responsive and -nonresponsive cancer cell xenografts.

These studies provide direct evidence for in vivo inhibitory AhR-AR crosstalk and determine the effectiveness of SAhRMs as indirect antiandrogens for treatment for androgen-responsive prostate cancer.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Boasberg, et al., Proc. Am. Soc. Clin. Oncol. 16:1126, 1997.
Boccardo, Crit. Rev. Oncol. Hematol. 35:121-132, 2000.
Cheng, et al., In: Prostate Diseases, edited by H. Lepor and R. K. Lawson. 1993, p. 57-71.
Chiarodo, Cancer Res. 51:2498-2505, 1991.
Dorai, et al., Prostate 32:246-258, 1997.
Folkman, N. Engl. J. Med. 285:1182-1186, 1971.
Folkman, Nat. Med. 1:27-31, 1995.
Fuqua, et al., Endocr. Relat. Cancer 2:19-25, 1995.
Gaido et al., Mol. Pharmacol. 58:852-858, 2000.
Gao, et al., Prostate 31:264-281, 1997.
Gross, et al., J. Urol. 159:2035-2039, 1998.
Hopp and Fuqua, J. Mammary Gland Biol. and Neoplasia 3:73-83, 1999.
Kelly, et al., Proc. Am. Soc. Clin. Oncol. 15:250, 1996.
Klotz, Cancer 88:3009-3014, 2000.
Knox, et al., Prostate 35:248-254, 1998.
Kubota, et al., Cancer Res. 58:3344-3352, 1998.
Landis, et al., Cancer J. Clin. 48:6-29, 1998.
McDougal et al., Cancer Res. 61:3901-3907, 2001
Miller, et al., Cancer Res. 52:515-520, 1992.
Miller, et al., Clin. Cancer Res. 1:997-1003, 1995.
Morris and Scher, Cancer 89:1329-1348, 2000.
Peehl, et al., Cancer Res. 54:805-810, 1994.
Pienta, et al., Cancer Res. 53:224-226, 1993.
Pollard, et al., Cancer Res. 51:3610-3611, 1991.
Sadar, et al., Endocr. Relat. Cancer 6:487-502, 1999.
Sakti and Crawford. Cancer of the Prostate. New York: Marcel Dekker, 1993. p. 1.
Shibata, et al., J. Natl. Cancer Inst. 90:1230-1231, 1998.
Smith, et al., Proc. Am. Soc. Clin. Oncol. 18:328, 1999.
Tontonoz, et al., Cell 79:1147-1156, 1994.
Tontonoz, et al., Proc. Natl. Acad. Sci. U.S.A. 94:237-241, 1997.
Wang, et al., Arch. Biochem. Biophys. 356:239-248, 1998.
Yamaoka, et al., Cancer Res. 53:5233-5236, 1993.

Although the present invention and its advantages have been described in detail, it should be understood that various changes, substitutions and alterations can be made herein without departing from the spirit and scope of the invention as defined by the appended description. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended descriptions are intended to include within their scope such processes, machines, manufacture, compositions of matter, means, methods, or steps.

1. A method of modulating a prostate neoplasm comprising administering to a subject an effective amount of a compound of the formula: 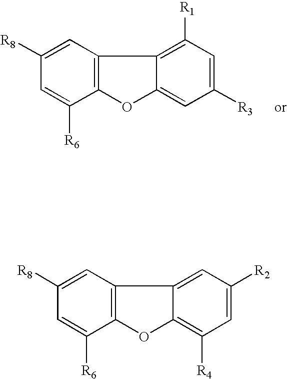
wherein R₁, R₃, R₆ and R₈ or R₂, R₄, R₆ and R₈ are individually and independently a hydrogen or a substituent selected from the group consisting of chlorine, fluorine, and bromine, and a linear or branched alkyl group of one to four carbons, said compound having at least one alkyl substituent and at least two halogen substituents.

2. The method of claim 1, wherein the halogen substituents are selected from the group consisting of chlorine, bromine, and fluorine.

3. The method of claim 1, wherein said alkyl substituents are selected from the group consisting of methyl, ethyl and propyl.

4. The method of claim 1, wherein the compound is 6-methyl-1,3,8-trichlorodibenzofuran.

5. The method of claim 1, wherein the compound is 8-methyl-1,3,6-trichlorodibenzofuran.

6. The method of claim 1, wherein the compound is 6-propyl-1,3,8-trichlorodibenzofuran.

7. The method of claim 1, wherein the compound is selected from the group consisting of 6-methyl-1,3,8-trichlorodibenzofuran, 8-methyl-1,3,6-trichlorodibenzofuran, 6-ethyl-1,3,8-trichlorodibenzofuran, 6-propyl-1,3-8-trichlorodibenzofuran, 6-methyl-2,3,8-trichlorodibenzofuran, 6-methyl-2,3,4,8-tetrachlorodibenzofuran, 8-methyl-1,3,7-trichlorodibenzofuran, 8-methyl-1,2,4,7-tetrachlorodibenzofuran, 8-methyl-2,3,7-trichlorodibenzofuran, and 8-methyl-2,3,4,7-tetrachlorodibenzofuran.

8. The method of claim 1, wherein the neoplasm is androgen responsive.

9. The method of claim 1, wherein the neoplasm is androgen non-responsive.

10. The method of claim 1, wherein the neoplasm is benign.

11. The method of claim 1, wherein the neoplasm is malignant.

12. A method of treating androgen-dependent tumors comprising administering to a subject in need of such treatment an effective amount 6-methyl-1,3,8-trichlorodibenzofuran.

13. A method of treating androgen-independent tumors comprising administering to a subject in need of such treatment an effective amount 6-methyl-1,3,8-trichlorodibenzofuran.
